(19) United States
(12) Patent Application Publication
(10) Pub. No.: US 2007/0154888 A1
(43) Pub. Date: Jul. 5, 2007

(54) Method for the covalent immobilization of probe biomolecules on organic surfaces

(76) Inventor: Holger Klapproth, Kehlerstrasse 12, Freiberg (DE)

Correspondence Address: THE WEBB LAW FIRM, P.C., 700 KOPPERS BUILDING, 436 SEVENTH AVENUE, PITTSBURGH, PA, 15219, US

(21) Appl. No.: 10/557,878

(22) PCT Filed: May 24, 2004
(86) PCT No.: PCT/DE04/01083

§ 371 Date: Oct. 3, 2006

(30) Foreign Application Priority Data
May 22, 2003 (DE) 103 23 685.6

(51) Int. Cl. C12Q 1/68; C12M 3/00; B05D 3/02; C07H 21/04

(52) U.S. Cl. 435/6; 435/287.2; 427/2.11; 536/25.32

(57) ABSTRACT

The invention relates to a method for the covalent immobilization of probe biomolecules on organic surfaces, by means of photoreactive cross-linking agents which are used to covalently immobilize the probe biomolecules on organic surfaces, or to covalently bind said probe biomolecules to soluble polymers or copolymers that are then covalently immobilized on an organic surface.

The invention relates to a method for the covalent immobilization of probe biomolecules on organic surfaces such as polymer surfaces or surfaces of inorganic substrates modified with self-assembled monolayers with photoreactive cross-linking agents that are used to covalently immobilize the probe biomolecules on an organic surface, or to covalently bind said probe biomolecules to soluble polymers or copolymers that are then covalently immobilized on an organic surface.

Microtechniques have become increasingly important in analysis in recent years and there are numerous solid phase systems developed on the basis of self-assembled monolayers (SAMs) of linkers, by means of which probe molecules specifically are bound to or conjugated with the surface of the solid carrier, where they can then be detected with the help of suitable markers (exemplarily radioactive, colored, fluorescent).

Analogous to electronic microchips, the designation sensor chips has also come into use for these systems. The term biochips is also used in reference to the conjugation of biological molecules. (known as bioconjugation) on such sensor chips, which are exemplarily oligonucleotides or antibodies. The binding to the carrier surface may be direct or indirect. An exemplary indirect binding is the binding of a nucleic acid sequence to be detected by means of hybridization on an immobilized, complementary oligonucleotide as a probe. In this case, using the probe has the additional advantage of the natural specificity of the interaction of biological macromolecules.

Typically in the production of sensor chips, metal or semi-metal oxides, for example aluminum oxide, quartz glass, or glass surfaces are immersed in a solution of linkers which for example have a halogen silicate (e.g. silicon chloride) or alkoxyl silicate group to bind to the carrier surface, which results in the formation of a self-assembled monolayer (SAM). In this case, said layer is only a few angstroms thick. The linker is bound to the probe or tracer molecule by means of an additional suitable functional group, for example an amino- or an epoxy group. Suitable linkers for bonding numerous probe or tracer molecules, especially those of biological origin, to numerous carrier surfaces are well-known to one skilled in the art; see for example “Bioconjugate Techniques” by G. T. Hermanson, Academic Press 1996.

A disadvantage of these reactive (and therefore sensitive) surfaces, which are exemplarily surfaces with epoxy, aldehyde or amino functions, is their often limited stability in storage (a few weeks), which requires them to be stored hermetically sealed.

The immobilization of e.g., nucleic acids on non-reactive polymer or synthetic/plastic surfaces (e.g., as probes for the production of sensor/biochips) with traditional methods is complicated and requires considerable effort.

The object of the invention is therefore the preparation of a method for the immobilization of probe biomolecules on organic surfaces, e.g., polymer surfaces or inorganic substrates modified by organic substances, which is both fast and easy to use.

This object is solved in the invention by a method of covalent immobilization of probe biomolecules on organic surfaces, exemplarily polymer surfaces, in which (a) one or a plurality photo cross-linkers (these may be bonded terminally or laterally or form an integral component of the chain of the biomolecule) are directly bound to a probe biomolecule, or indirectly across a spacer, and (b) the reaction product from (a) is superimposed on an exemplary polymer surface (e.g. by the exertion of pressure) and covalently bonded to said polymer surface by irradiation with light of a proper wavelength (e.g., UV light), or the reaction product from (a) is bound to a soluble (e.g., water soluble) polymer or copolymer, which is then immobilized on a surface composed of organic molecules, for example a polymer surface.

This object is alternatively solved by a method of covalent immobilization of probe biomolecules on organic surfaces which are for example polymer surfaces, wherein
(a) a soluble (e.g., water soluble) polymer or copolymer with reactive groups is produced, and after the polymerization oligomers and polymers are covalently bonded with one or a plurality of photoreactive groups (the photoreactive group can be bonded terminally or laterally or form an integral component of the chain of the biomolecule) and probes or receptor biomolecules (to which a target biomolecule to be detected can bind),
or
b) wherein the soluble polymer or copolymer is copolymerized with reactive group-forming monomers, copolymerizable monomers are copolymerized with one or a plurality of photoreactive groups and copolymerizable probe biomolecules are copolymerized (by the casserole method),
c) the reaction product from (a) or (b) is superimposed on an organic surface (e.g., by exerting pressure) and covalently immobilized on said surface by irradiation with light of a proper wave length (e.g., UV light).

The advantage of the invention is that it is possible to press a viscous medium; exemplarily the reaction product of steps (a) or (b) of the alternative method described above that is very easy to immobilize, namely by irradiation with light of a proper wavelength, onto non-reactive surfaces (e.g., silicate glass carriers or substrates made of commercially. available synthetic materials). Because this method also results in the construction of a pseudo three dimensional matrix, the quantity of analyte that can be bound is likewise substantially increased. In addition, this method solves the classic problems associated with three dimensional matrices, such as the displacement effect of the medium when pressure is exerted on polymer gels. Furthermore, it is not necessary to exert pressure on reactive (and therefore sensitive) surfaces. Exemplary reactive surfaces are those with epoxy, aldehyde or amino functional groups. Reactive surfaces often have limited stability (a few weeks) and must be stored hermetically sealed. Not having a reactive surface means that carriers of e.g., polystyrole or polymethylmethacrylate (PMMA), which remain stable for years, may be used. An additional advantage is that e.g., polymer surfaces do not have to be hydrophilized by preliminary processing steps such as plasma processing, because the surface in the exemplary alternative embodiment of the method of the invention described above is made accessible by means of the bound (swellable, wettable) copolymer. Apart from this, the surface characteristics of the substrate (exemplarily the sensor surface) may be checked readily and very accurately. An exemplary important surface characteristic that is readily checked with the aid of the method described herein is wettability. An additional advantage is the simplified analysis, as in principle only the volume of the superimposed drop must be defined and the number of immobilized probes is then derived directly therefrom. This procedure, for example, for binding DNA to SAMs, is not trivial from the standpoint of the state-of-the-art.

The invention further relates to an organic surface that is exemplarily a polymer surface with probe molecules covalently immobilized thereon, preferably forming a pattern (e.g., by exerting pressure), which surface can be obtained according to a method defined above.

The invention further relates to the use of an organic surface, for example a polymer surface with probe biomolecules immobilized thereon and preferably forming a pattern, as a sensor chip; moreover, in an additional embodiment, it relates to a medical or diagnostic instrument that has an organic surface of the invention such as a polymer surface or a sensor chip contained therein.

Advantageous and/or preferred embodiments of the invention are objects of the subordinate claims.

In the method of the invention, the photoreactive group(s) can be chosen from benzophenone or its derivatives, anthrachinone or its derivatives and thymidine or its derivatives.

Exemplary suitable reactive groups are epoxy, carboxy, active ester, isocyanate, maleinimide, isothiocyanate and azlactone groups.

According to an exemplary embodiment of the alternate method of the invention, in step (a) the soluble polymer or copolymer is synthesized with reactive groups by means of copolymerization of (meth)acrylic acid and/or dimethylacrylamide and/or vinylpyrrolidone and glycidylmethacrylate.

According to an additional embodiment of the alternate method of the invention, in step (a) the photoreactive oligomeres or polymers are formed by the covalent bonding of 5′ amino-modified oligothymidine, and the probe biomolecule is formed by the covalent bonding of 5′ amino-modified probe molecules.

A primary amino group may be modified in the amino-modification. It must be mentioned here, however, that the photoreactive oligomers or polymers and the probe or receptor molecule in no way have to be modified in the same manner, e.g., 5′amino-modified, in order to be covalently bonded to the soluble polymer or copolymer. This makes the alternative method of the invention especially easy to use. The group used for the modification is not subject to any special restrictions; rather, it is selected in relation to the given conditions. A carboxy or thio-modification is also an exemplary possibility.

According to an additional embodiment of the alternative method of the invention, in step (b) 5′-aryl modified oligothymidine and 5′-aryl or 3′-aryl modified probe biomolecules are copolymerized with one or a plurality of acrylate(s) or methacrylate(s).

According to an additional embodiment of the alternative method of the invention, in step (b) 4-methacryoyloxybenzophenone and 5′-aryl or 3′-modified probe biomolecules are copolymerized with one or a plurality of acrylate(s) or methacrylate(s).

The photoreactive group(s) is/are exemplarily ultraviolet reactive.

According to additional embodiments of the method of the invention, the probe biomolecules with directly or indirectly bound photoreactive groups or (in the alternative method) the soluble polymer or copolymer with covalent bonded photoreactive oligomers or polymers and probe biomolecules are superimposed forming a pattern on an organic surface which is for example, a polymer surface.

For the method of the invention, exemplary polymer surfaces made of cycloolefincopolymers (COCs), polystyrole, polyethylene, polypropylene or polymethylmethacrylate (PMMA, Plexiglas) are suitable as organic surfaces. A suitable COC, for example, is marketed by Ticona under the trade name “Topas.” It must be expressly mentioned here that the method of the invention is suitable for any organic surface in relation to the photoreactive groups used. Surfaces coated with organic molecules such as inorganic substrates coated with self-assembled monolayers (SAMs) are also suitable for this purpose. These SAMs themselves may be completely inert and thus may exemplarily consist purely of alkylsilicates.

In the method of the invention, the probe biomolecule may exemplarily be a partner of a specific complementary bonding partner interacting system (receptor/ligand).

A specific complementary bonding partner interaction system may be based on the interaction of a nucleic acid with a complementary nucleic acid, the interaction of a peptide nucleic acid (PNA) with a nucleic acid, the enzyme/substrate, receptor effector, lectin/sugar, antibody/antigen, avidin/biotin or streptavidin/biotin interaction.

The nucleic acids obviously can be either DNA or RNA, exemplarily an oligonucleotide or an aptamer or also a so-called “LNA” such as that offered at www.proligo.com or also a monopolymerizable DNA such as that offered under the trade name “Acrydite” at www.mosaic-technologies.com. Peptide nucleic acids (PNAs) are also an option.

The exemplary antibody may be a polyclonal, monoclonal, chimaric or single chain antibody or a functional fragment or derivative (by functional it is meant that the fragment/derivative can bind to an antigen without immunogenity) of such an antibody.

In the following, the invention will be explained in more detail with reference to, but not limited to concrete embodiments and examples and with reference to nucleic acids as probe biomolecules.

Synthesis of the Copolymer:

An exemplary suitable copolymer can be produced by means of the copolymerization of methacrylic acid and glycidylmethacrylate in a 1:20 (mol/mol) mixture by the addition of 1% AIBN (azobisiso-butyronitrile) to a solution of monomers in a suitable solute (e.g., 10% (v/v) monomers in chloroform). The resulting copolymer can then be precipitated out with diethylether.

A photoreactive side group can exemplarily be inserted by adding 5′-amino-modified oligothymidine. At this point, amino-modified nucleic acids such as DNA can be selectively bonded to glycidyl remnants that have not been used up in the reaction or added simultaneously with the oligothymidine so that a competition reaction takes place between the oligothymidine and the nucleic acid/DNA. The amino-modified nucleic acid/DNA can exemplarily be bound to the polymer in an aqueous sodium phosphate solution with a pH of 9.

The copolymer substituted in this manner can now be analyzed (to determine the DNA content) and be pressed onto almost any organic polymer surface as a substrate. The polymer is immobilized by UV irradiation at 260 nm.

In another approach, a copolymer is formed from a monomer with a UV reactive group, a reactive monomer and a hydrophilic (inert) monomer; e.g., 4-methacryloyloxybenzophenone, glycidoxymethacrylate and methacrylic acid. A 50 nm thick layer on a PMMA substrate is formed from this polymer. In this case the polymer is immobilized exclusively by a photo-induced bonding reaction between the benzophenone groups contained in the polymer and the substrate, triggered by UV irradiation at 300 nm.

1. A method of covalent immobilization of probe biomolecules on organic surfaces, wherein
(a) one or a plurality of photoreactive groups is attached directly, or indirectly across a spacer to a probe biomolecule, and
(b) the reaction product from (a) is superimposed on an organic surface and covalently immobilized on said surface by irradiation with light of a proper wavelength, or the reaction product from (a) is covalently attached to a soluble polymer or copolymer, which polymer or copolymer is then covalently immobilized on an organic surface.

2. A method of covalent immobilization of probe biomolecules on organic surfaces, wherein
(a) a soluble polymer or copolymer with reactive groups is synthesized and oligomers or polymers with one or a plurality of photoreactive groups and probe biomolecules are covalently attached following polymerization, or
(b) wherein the soluble polymer or copolymer is copolymerized with reactive group-forming monomers, copolymerizable monomers are copolymerized with one or a plurality of photoreactive groups, and copolymerizable probe biomolecules are copolymerized,
(c) the reaction product from (a) or (b) is superimposed on an organic surface and covalently immobilized thereon by irradiation with light of a proper wavelength.

3. A method as defined in claim 1, in which one or a plurality of groups chosen from benzophenone or its derivatives, anthrachinone or its derivatives and thymidine or its derivatives is used as the photoreactive group(s).

4. A method as defined in claim 2, wherein the soluble polymer or copolymer with reactive groups is synthesized in step (a) by means of copolymerization of (meth)acrylic acid and/or dimethylacrylamide and/or vinyl pyrrolidone and glycidylmethacrylate.

5. A method as defined in claim 4, wherein the photoreactive oligomers or polymers are formed in step (a) by covalent attaching of 5′-amino-modified oligothymidine and the probe biomolecules are formed in step (a) by covalent attaching of 5′-amino-modified probe biomolecules.

6. A method as defined in claim 2, wherein 5′-aryl-modified oligothymidine and 5′-aryl-or 3′-modified probe biomolecules are copolymerized in step (b) as comonomers with one or a plurality of acrylates or methacrylates.

7. A method as defined in claim 2, wherein 4-methacryloyloxybenzophenone and 5′-aryl or 3′-modified probe biomolecules are copolymerized in step (b) as comonomers with one or a plurality of acrylates or methacrylates.

8. A method as defined in claim 1, wherein the photoreactive group(s) is/are ultraviolet reactive.

9. A method as defined in claim 1, wherein the superimposition is achieved by exerting pressure forming a pattern in step (b) defined in claim 1 or in step (c) defined in claim 2.

10. A method as defined in claim 1, wherein the polymer surface is composed of cycloolefincopolymerene, poly-styrole, polyethylene, polypropylene or polymethylmethacrylate.

11. A method as defined in claim 1, wherein a partner of a specific interaction system of complementary attaching partners (receptor/ligand) is used as a probe biomolecule.

12. A method as defined in claim 11, wherein the specific complementary attaching partner interaction system is based on the interaction of a nucleic acid with a complementary nucleic acid, the interaction of a peptide nucleic acid with a nucleic acid, the enzyme/substrate, receptor/effector, lectin/sugar, antibody/antigen, avidin/biotin or streptavidin/biotin interaction.

13. A method as defined in claim 12, wherein the nucleic acid is DNA or RNA or an analog thereof.

14. A method as defined in claim 13, wherein the DNA or the RNA is an oligonucleotide.

15. A method as defined in claim 12, wherein the antibody is a polyclonal, monoclonal, chimaric or single chain antibody or a functional fragment or a derivative of such an antibody.

16. An organic surface with covalently immobilized probe biomolecules thereon that can be obtained by a method as defined in claim 1.

17. An organic surface with covalently immobilized probe molecules forming a pattern thereon that can be obtained by a method as defined in claim 1.

18. A use of an organic surface as defined in claim 17 as a sensor chip.

19. A medical or diagnostic instrument having an organic surface as defined in claim 16.
